(19) United States
(12) Patent Application Publication
(10) Pub. No.: US 2024/0268929 A1
(43) Pub. Date: Aug. 15, 2024

(54) DATA PROCESSING APPARATUS FOR PROCESSING ORAL IMAGE AND ORAL IMAGE PROCESSING METHOD

(71) Applicant: MEDIT CORP., Seoul (KR)

(72) Inventor: Dong Hoon LEE, Seoul (KR)

(73) Assignee: MEDIT CORP., Seoul (KR)

(21) Appl. No.: 18/568,481

(22) PCT Filed: Jun. 9, 2022
(86) PCT No.: PCT/KR2022/008106

§ 371 Date: Dec. 8, 2023

(30) Foreign Application Priority Data
Jun. 9, 2021 (KR) 10-2021-0074971
Jun. 8, 2022 (KR) 10-2022-0069702

(51) Int. Cl. A61C 7/00; A61C 9/00; G06T 19/20; G16H 30/40

(52) U.S. Cl. CPC A61C 7/002; A61C 9/0053; G06T 19/20; G16H 30/40

(57) ABSTRACT

Provided are a data processing apparatus for processing an intraoral image and an intraoral image processing method. An intraoral image processing method of a data processing apparatus includes obtaining upper-jaw scan data obtained by scanning an upper jaw of an oral cavity, lower-jaw scan data obtained by scanning a lower jaw of the oral cavity, and occlusion scan data obtained by scanning an occlusion state of the upper jaw and the lower jaw, selecting a mobility tooth, and performing occlusion alignment of the upper-jaw scan data and the lower-jaw scan data based on the occlusion scan data excluding the selected mobility tooth.

### TECHNICAL FIELD

Described embodiments relate to a data processing apparatus for processing an intraoral image and an intraoral image processing method.

Particularly, the described embodiments relate to a data processing apparatus and an intraoral image processing method for reliably occlusion-aligning one or more teeth included in an intraoral image.

### BACKGROUND ART

For dental treatment of a patient, an intraoral scanner may be inserted into an oral cavity (mouth) of the patient and an intraoral image of the patient may be obtained by the intraoral scanner. The intraoral image obtained by the intraoral scanner may include one or more teeth. For a process such as prosthetic treatment or orthodontic treatment that is dental treatment, it is necessary to occlusally align the teeth included in the intraoral image. Moreover, there may be a loose tooth (hereinafter referred to as a ‘mobility tooth’) among the teeth included in the patient's oral cavity. Because the tooth position of the mobility tooth may move due to an occlusion force during an occlusion scan, occlusion alignment may not be well achieved due to the difference between occlusion scan data and upper-jaw/lower-jaw (maxillary/mandibular) scan data, or even when occlusion alignment is achieved, because the occlusion alignment is achieved based on data including a moving tooth, unreliable occlusion data may be obtained. Thus, there is a need for a method of reliably performing occlusion alignment even when a mobility tooth is included in the patient's oral cavity.

### DISCLOSURE

### Technical Problem

The described embodiments provide an intraoral image processing method of reliably occlusion-aligning one or more teeth included in an intraoral image, and an apparatus for performing an operation according thereto.

### Technical Solution

According to an embodiment, an intraoral image processing method of a data processing apparatus includes obtaining upper-jaw scan data obtained by scanning an upper jaw of an oral cavity, lower-jaw scan data obtained by scanning a lower jaw of the oral cavity, and occlusion scan data obtained by scanning an occlusion state of the upper jaw and the lower jaw, selecting a mobility tooth, and performing occlusion alignment of the upper-jaw scan data and the lower-jaw scan data based on the occlusion scan data excluding the selected mobility tooth.

According to an embodiment, the selecting of the mobility tooth may include selecting the mobility tooth according to a user input received through a user interface.

According to an embodiment, the performing of the occlusion alignment may include individualizing teeth of the upper-jaw scan data and teeth of the lower-jaw scan data, recognizing the selected mobility tooth from the individualized teeth, and performing occlusion alignment of the upper-jaw scan data and the lower-jaw scan data by using the occlusion scan data excluding the recognized mobility tooth.

According to an embodiment, the selecting of the mobility tooth may include obtaining occlusion alignment upper-jaw scan data and occlusion alignment lower-jaw scan data by performing occlusion alignment of the upper-jaw scan data and the lower-jaw scan data by using the occlusion scan data, and selecting the mobility tooth from the occlusion alignment upper-jaw scan data and the occlusion alignment lower-jaw scan data, and the performing of the occlusion alignment may include performing occlusion realignment of the occlusion alignment upper-jaw scan data and the occlusion alignment lower-jaw scan data by using the occlusion scan data excluding the selected mobility tooth.

According to an embodiment, the selecting of the mobility tooth from the occlusion alignment upper-jaw scan data or the occlusion alignment lower-jaw scan data may include selecting the mobility tooth based on a user input for selecting the mobility tooth through a user interface with the occlusion alignment upper-jaw scan data or the occlusion alignment lower-jaw scan data displayed therein, or automatically selecting the mobility tooth by the data processing apparatus.

According to an embodiment, a data processing apparatus includes a processor and a memory, wherein the processor executes one or more instructions stored in the memory to obtain upper-jaw scan data obtained by scanning an upper jaw of an oral cavity, lower-jaw scan data obtained by scanning a lower jaw of the oral cavity, and occlusion scan data obtained by scanning an occlusion state of the upper jaw and the lower jaw, select a mobility tooth, and perform occlusion alignment of the upper-jaw scan data and the lower-jaw scan data by using the occlusion scan data excluding the selected mobility tooth.

According to an embodiment, a computer-readable recording medium has recorded thereon a program implemented to execute an intraoral image processing method by a processor of a data processing apparatus, wherein the intraoral image processing method includes obtaining upper-jaw scan data obtained by scanning an upper jaw of an oral cavity, lower-jaw scan data obtained by scanning a lower jaw of the oral cavity, and occlusion scan data obtained by scanning an occlusion state of the upper jaw and the lower jaw, selecting a mobility tooth, and performing occlusion alignment of the upper-jaw scan data and the lower-jaw scan data by using the occlusion scan data excluding the selected mobility tooth.

### Advantageous Effects

The intraoral image processing method and the apparatus for performing an operation according thereto according to the described embodiments may perform more reliable occlusion alignment by identifying a mobility tooth among the teeth included in an intraoral image and excluding the identified mobility tooth in an occlusion alignment operation.

### DESCRIPTION OF DRAWINGS

The present disclosure may be easily understood through the following detailed description and the accompanying drawings, in which reference numerals refer to structural elements.

FIG. 1 is a diagram for describing an intraoral image processing system according to described embodiments.

FIG. 2 is a block diagram illustrating a data processing apparatus according to described embodiments.

FIG. 3 is a flowchart illustrating a process of a method of performing occlusion alignment of scan data, according to an embodiment.

FIG. 4 illustrates upper-jaw scan data, lower-jaw scan data, and occlusion scan data according to an example.

FIG. 5 is a reference diagram for describing an example of performing occlusion alignment excluding a mobility tooth, according to an embodiment.

FIG. 6 is a flowchart illustrating an example of a process of a method of performing occlusion alignment of scan data, according to an embodiment.

FIG. 7 illustrates an example of a user interface for selecting a mobility tooth, according to an embodiment.

FIG. 8 is a reference diagram for describing a method of individualizing teeth, according to an embodiment.

FIG. 9 is a flowchart illustrating an example of a process of a method of performing occlusion alignment of scan data, according to an embodiment.

FIG. 10 is a flowchart illustrating an example of a process of a method of performing occlusion alignment of scan data, according to an embodiment.

### MODE FOR INVENTION

The specification clarifies the scope of the present disclosure and describes the principle of the present disclosure and embodiments so that those of ordinary skill in the art may implement the present disclosure. The described embodiments may be implemented in various forms.

Throughout the specification, like reference numerals will denote like elements. The specification may not describe all elements of the embodiments, and general descriptions in the art to which the present disclosure belongs or redundant descriptions between the embodiments will be omitted for conciseness. The term ‘unit’ (or part or portion) used herein may be implemented as software or hardware, and depending on the embodiments, a plurality of ‘units’ may be implemented as one element (or unit) or one ‘unit’ may include a plurality of elements. Hereinafter, the operation principle and embodiments of the present disclosure will be described with reference to the accompanying drawings.

Herein, an image may include an image representing at least one tooth or an oral cavity (mouth) including at least one tooth or an image representing a tooth model (hereinafter referred to as an ‘intraoral image’).

Also, herein, the image may be a two-dimensional (2D) image of an object or a three-dimensional (3D) model or a 3D image stereoscopically representing an object. Also, herein, the image may refer to data required to represent an object in 2D or 3D, for example, raw data obtained from at least one image sensor. Particularly, the raw data may be 2D or 3D data obtained to generate an intraoral image and may be data (2D data in an embodiment) obtained from at least one image sensor included in an intraoral scanner when the inside of the oral cavity of a patient as an object is scanned by using a 3D scanner. The 3D scanner may include an intraoral scanner, a table scanner, a CT scanner, and the like.

Hereinafter, an intraoral scanner will be described as an example.

Herein, the ‘object’ may include a tooth, a gum, at least some area of the oral cavity, and/or an artificial structure insertable into the oral cavity (e.g., an orthodontic device, an implant, an artificial tooth, or an orthodontic assistance tool inserted into the oral cavity). Here, the orthodontic device may include at least one of a bracket, an attachment, an orthodontic screw, a lingual orthodontic device, and a removable orthodontic retainer.

Hereinafter, embodiments will be described in detail with reference to the accompanying drawings.

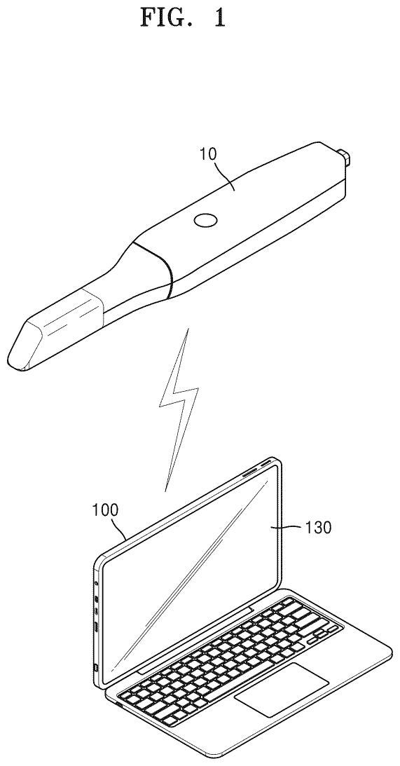

FIG. 1 is a diagram for describing an intraoral image processing system according to described embodiments.

Referring to FIG. 1, an intraoral scanner 10 may be a medical device for obtaining an image of the inside of an oral cavity.

Particularly, the intraoral scanner 10 may be a device for obtaining an image of the oral cavity including at least one tooth by being inserted into the oral cavity and scanning teeth in a non-contact manner. Also, the intraoral scanner 10 may have a form capable of being inserted into and withdrawn from the oral cavity and may scan the inside of the patient's oral cavity by using at least one image sensor (e.g., an optical camera). In order to image the surface of at least one of a tooth and a gum inside the oral cavity and an artificial structure insertable into the oral cavity (e.g., an orthodontic device including brackets and wires, an implant, an artificial tooth, or an orthodontic assistance tool inserted into the oral cavity) which is an object, the intraoral scanner 10 may obtain surface information about the object as raw data. Also, the intraoral scanner 10 may obtain an intraoral image not only by directly scanning the patient's oral cavity but also by scanning a tooth model or a plaster model obtained by taking an impression of the patient.

The image data obtained by the intraoral scanner 10 may be transmitted to a data processing apparatus 100 connected through a wired or wireless communication network.

The data processing apparatus 100 may be any electronic apparatus that may be connected to the intraoral scanner 10 through a wired or wireless communication network, may receive a 2D image obtained by scanning the oral cavity from the intraoral scanner 10, and may generate, process, display, and/or transmit an intraoral image based on the received 2D image. In addition to receiving the intraoral image obtained by the intraoral scanner 10, the data processing apparatus 100 may also receive, from a 3D model scanner, a tooth model image obtained by the 3D model scanner by scanning a tooth model.

Based on the 2D image data received from the intraoral scanner 10, the data processing apparatus 100 may generate at least one of information generated by processing the 2D image data and an intraoral image generated by processing the 2D image data and may display the generated information and intraoral image through a display 130.

In FIG. 1, the data processing apparatus 100 is illustrated in the form of a laptop computer; however, the present disclosure is not limited thereto, and the data processing apparatus 100 may be any computing device such as a smart phone, a desktop computer, a PDA, or a tablet PC but is not limited thereto.

Also, the data processing apparatus 100 may be provided in the form of a server (or a server device) for processing an intraoral image.

Also, the intraoral scanner 10 may intactly transmit, to the data processing apparatus 100, the raw data obtained through an intraoral scan. In this case, based on the received raw data, the data processing apparatus 100 may generate a 3D intraoral image representing the oral cavity in 3D. Also, because the ‘3D intraoral image’ may be generated by 3D modeling of the internal structure of the oral cavity based on the received raw data, it may also be referred to as a ‘3D intraoral model’ or a ‘3D intraoral image’. Hereinafter, a model or an image representing the oral cavity in 2D or 3D will be collectively referred to as an ‘intraoral image’.

Also, the data processing apparatus 100 may analyze, process, display, and/or transmit the generated intraoral image to an external device.

As another example, the intraoral scanner 10 may obtain raw data through an intraoral scan, generate an image corresponding to the oral cavity as an object by processing the obtained raw data, and transmit the image to the data processing apparatus 100. In this case, the data processing apparatus 100 may analyze, process, display, and/or transmit the received image.

Although the intraoral scanner 10 is illustrated as a device for providing scan data to the data processing apparatus 100 in the system illustrated in FIG. 1, the described embodiments are not limited thereto and a table scanner for scanning a plaster model or the like may also be used.

In the system illustrated in FIG. 1, when a user such as a doctor scans an upper jaw (maxilla) and a lower jaw (mandible) of the patient's oral cavity and an occlusion state of the upper jaw and the lower jaw by using the intraoral scanner 10, the intraoral scanner 10 may provide, to the data processing apparatus 100, upper-jaw (maxillary) scan data obtained by scanning the upper jaw of the patient, lower-jaw (mandibular) scan data obtained by scanning the lower jaw, and occlusion scan data obtained by scanning the occlusion state. The data processing apparatus 100 may obtain data used for dental treatment of the patient by processing the upper-jaw scan data, the lower-jaw scan data, and the occlusion scan data received from the intraoral scanner 10. The data processing apparatus 100 may obtain occlusally-aligned scan data by aligning the upper-jaw scan data and the lower-jaw scan data by using the occlusion scan data.

In this case, when there is a mobility tooth in an oral cavity of the patient, because a mobility tooth image included in the upper-jaw scan data or the lower-jaw scan data obtained by scanning the oral cavity may not represent the accurate position of the tooth, reliable occlusion alignment results may not be obtained when the mobility tooth image is intactly used for occlusion alignment. Thus, the data processing apparatus 100 may improve the reliability of an occlusion alignment operation by identifying a mobility tooth and performing occlusion alignment excluding the identified mobility tooth when aligning the upper-jaw scan data and the lower-jaw scan data by using the occlusion scan data.

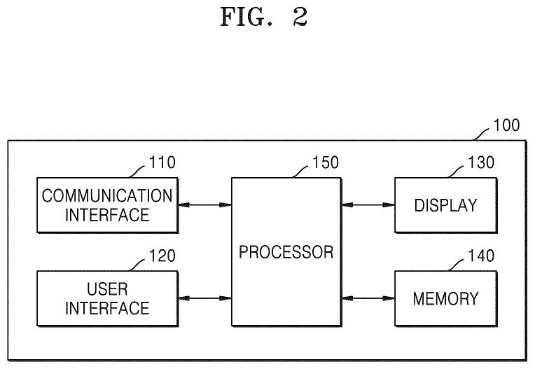

FIG. 2 is a block diagram illustrating a data processing apparatus 100 according to described embodiments.

Referring to FIG. 2, the data processing apparatus 100 may include a communication interface 110, a user interface 120, a display 130, a memory 140, and a processor 150.

The communication interface 110 may communicate with at least one external electronic device through a wired or wireless communication network. Particularly, the communication interface 110 may communicate with the intraoral scanner 10 under the control by the processor 150. Under the control by the processor 150, the communication interface 110 may communicate with an external electronic device or server or the like connected through a wired or wireless communication network.

The communication interface 110 may communicate with an external electronic device (e.g., an intraoral scanner, a server, or an external medical device) through a wired or wireless communication network. Particularly, the communication interface may include at least one short-range communication module for performing communication according to the communication standard such as Bluetooth, WiFi, Bluetooth Low Energy (BLE), NFC/RFID, WiFi Direct, UWB, or ZigBee.

Also, the communication interface 110 may further include a long-distance communication module for performing communication with a server to support long-distance communication according to the long-range communication standard. Particularly, the communication interface 110 may include a long-range communication module performing communication through a network for Internet communication. Also, the communication interface may include a long-range communication module performing communication through a communication network conforming to the communication standard such as 3G, 4G, and/or 5G.

Also, in order to communicate by wire with an external electronic device (e.g., an intraoral scanner), the communication interface 110 may include at least one port for being connected to the external electronic device through a wired cable. Accordingly, the communication interface 110 may perform communication with the external electronic device connected by wire through the at least one port.

The user interface 120 may receive a user input for controlling the data processing apparatus 100. The user interface 120 may include, but is not limited to, a user input device including a touch panel for sensing a user's touch, a button for receiving a user's push operation, and/or a mouse or a keyboard for designating or selecting a point on a user interface screen. Also, the user interface 120 may include a voice recognition device for voice recognition. For example, the voice recognition device may be a microphone, and the voice recognition device may receive a user's voice command or voice request. Accordingly, the processor may control an operation corresponding to the voice command or voice request to be performed.

According to an embodiment, the user interface 120 may receive a user input for selecting a mobility tooth from the user.

The display 130 may display a screen. Particularly, the display 130 may display a certain screen under the control by the processor 150. Particularly, the display 130 may display a user interface screen including the intraoral image generated based on the data obtained by scanning the patient's oral cavity by the intraoral scanner 10. Alternatively, the display 130 may display a user interface screen including information related to the patient's dental treatment.

The memory 140 may store at least one instruction. Also, the memory 140 may store at least one instruction executed by the processor 150. Also, the memory may store at least one program executed by the processor 150. Also, the memory 140 may store data received from the intraoral scanner (e.g., raw data obtained through intraoral scanning). Alternatively, the memory may store an intraoral image three-dimensionally representing the oral cavity.

According to an embodiment, the memory 140 may store upper-jaw scan data, lower-jaw scan data, and occlusion scan data in a state where the upper jaw and the lower jaw are occluded.

According to an embodiment, the memory 140 may include one or more instructions for performing an operation for occlusion alignment excluding the mobility tooth in order to reliably align the upper jaw and the lower jaw described herein.

The processor 150 may execute at least one instruction stored in the memory 140 to perform control such that a desired operation may be performed. Here, the at least one instruction may be stored in an internal memory included in the processor 150 or in the memory 140 included in the data processing apparatus separately from the processor.

Particularly, the processor 150 may execute at least one instruction to control at least one component included in the data processing apparatus such that a desired operation may be performed. Thus, although a case where the processor performs certain operations is described as an example, it may mean that the processor controls at least one component included in the data processing apparatus such that certain operations may be performed.

According to an embodiment, by executing one or more instructions stored in the memory 140, the processor 150 may obtain upper-jaw scan data obtained by scanning an upper jaw of an oral cavity, lower-jaw scan data obtained by scanning a lower jaw of the oral cavity, and occlusion scan data obtained by scanning an occlusion state of the upper jaw and the lower jaw, select a mobility tooth, and perform occlusion alignment of the upper-jaw scan data and the lower-jaw scan data by using the occlusion scan data excluding the selected mobility tooth.

According to an embodiment, by executing one or more instructions stored in the memory 140, the processor 150 may select the mobility tooth according to a user input received through a user interface.

According to an embodiment, by executing one or more instructions stored in the memory 140, the processor 150 may individualize teeth by automatically segmenting the upper-jaw scan data and the lower-jaw scan data, recognize the selected mobility tooth from the individualized teeth, and perform occlusion alignment of the upper-jaw scan data and the lower-jaw scan data by using the occlusion scan data excluding the recognized mobility tooth.

According to an embodiment, by executing one or more instructions stored in the memory 140, the processor 150 may obtain occlusion alignment upper-jaw scan data and occlusion alignment lower-jaw scan data by performing occlusion alignment of the upper-jaw scan data and the lower-jaw scan data by using the occlusion scan data, select the mobility tooth from the occlusion alignment upper-jaw scan data and the occlusion alignment lower-jaw scan data, and perform occlusion realignment of the occlusion alignment upper-jaw scan data and the occlusion alignment lower jaw scan data by using the occlusion scan data excluding the selected mobility tooth.

According to an embodiment, by executing one or more instructions stored in the memory 140, the processor 150 may select the mobility tooth from the occlusion alignment upper-jaw scan data or the occlusion alignment lower-jaw scan data and may select the mobility tooth based on a user input for selecting the mobility tooth through a user interface with the occlusion alignment upper-jaw scan data or the occlusion alignment lower-jaw scan data or automatically select the mobility tooth by the data processing apparatus.

According to an example, the processor 150 may internally include at least one internal processor and a memory device (e.g., RAM or ROM) for storing at least one of programs, instructions, signals, and data to be processed or used by the internal processor.

Also, the processor 150 may include a graphic processor (graphic processing unit) for graphic processing corresponding to video. Also, the processor may be implemented as a System-on-Chip (SoC) including a combination of a core and a GPU. Also, the processor may include a single core or a multi-core. For example, the processor may include a dual core, a triple core, a quad core, a hexa core, an octa core, a deca core, a dodeca core, a hexadecimal core, and/or the like.

In the described embodiments, the processor 150 may generate an intraoral image based on the 2D image received from the intraoral scanner 10.

Particularly, under the control by the processor 150, the communication interface 110 may receive the data obtained by the intraoral scanner 10, for example, the raw data obtained through intraoral scanning. Based on the raw data received by the communication interface, the processor 150 may generate a 3D intraoral image three-dimensionally representing the oral cavity. For example, in order to restore a 3D image according to an optical triangulation method, the intraoral scanner may include at least one camera and may include an L camera corresponding to the left field of view and an R camera corresponding to the right field of view in a particular embodiment. The intraoral scanner may obtain L image data corresponding to the left field of view and R image data corresponding to the right field of view from the L camera and the R camera, respectively. Subsequently, the intraoral scanner (not illustrated) may transmit raw data including the L image data and the R image data to the communication interface of the data processing apparatus 100.

Then, the communication interface 110 may transmit the received raw data to the processor, and the processor may generate an intraoral image three-dimensionally representing the oral cavity based on the received raw data.

Also, the processor 150 may control the communication interface to directly receive an intraoral image three-dimensionally representing the oral cavity from an external server, a medical device, or the like. In this case, the processor may obtain a 3D intraoral image without generating a 3D intraoral image based on the raw data.

According to the described embodiments, the processor 150 performing operations such as “extracting”, “obtaining”, and “generating” may include not only the processor 150 executing at least one instruction to directly perform the above operations but also the processor 150 controlling other components to perform the above operations.

In order to implement the embodiments described herein, the data processing apparatus 100 may include only some of the components illustrated in FIG. 2 or may include more components than the components illustrated in FIG. 2.

Also, the data processing apparatus 100 may store and execute dedicated software linked to the intraoral scanner. Here, the dedicated software may also be referred to as a dedicated program, a dedicated tool, or a dedicated application. When the data processing apparatus 100 operates in conjunction with the intraoral scanner 10, the dedicated software stored in the data processing apparatus 100 may be connected to the intraoral scanner 10 to receive the data obtained through intraoral scanning in real time. For example, there is dedicated software for processing data obtained through intraoral scanning by an intraoral scanner product of Medit. Particularly, Medit produces and distributes “Medit Link” as software for processing, managing, using, and/or transmitting data obtained by the intraoral scanner product. Here, because the “dedicated software” refers to a program, a tool, or an application operable in conjunction with the intraoral scanner, various intraoral scanners developed and sold by various manufacturers may be used in common. Also, the above dedicated software may be produced and distributed separately from the intraoral scanner for performing intraoral scanning.

The data processing apparatus 100 may store and execute dedicated software corresponding to the intraoral scanner product. The dedicated software may perform one or more operations for obtaining, processing, storing, and/or transmitting the intraoral image. Here, the dedicated software may be stored in the processor. Also, the dedicated software may provide a user interface for using the data obtained by the intraoral scanner. Here, the user interface screen provided by the dedicated software may include the intraoral image generated according to the described embodiments.

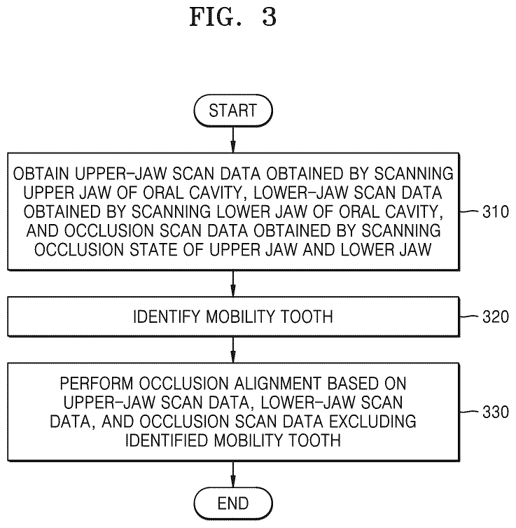

FIG. 3 is a flowchart illustrating a process of a method of performing occlusion alignment of scan data, according to an embodiment.

Referring to FIG. 3, in operation 310, the data processing apparatus 100 may obtain upper-jaw scan data obtained by scanning the upper jaw of the oral cavity, lower-jaw scan data obtained by scanning the lower jaw of the oral cavity, and occlusion scan data obtained by scanning an occlusion state of the upper jaw and the lower jaw. The data processing apparatus 100 may receive and obtain the upper-jaw scan data, the lower-jaw scan data, and the occlusion scan data in real time from the intraoral scanner 10 or may read the upper-jaw scan data, the lower-jaw scan data, and the occlusion scan data stored in the memory.

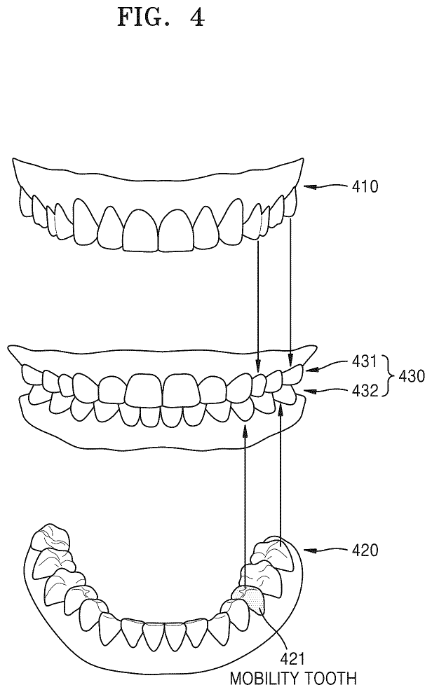

FIG. 4 illustrates upper-jaw scan data, lower-jaw scan data, and occlusion scan data according to an example.

Referring to FIG. 4, upper-jaw scan data 410 may represent scan data obtained by scanning the upper jaw of the patient's oral cavity, lower-jaw scan data 420 may represent scan data obtained by scanning the lower jaw of the patient's oral cavity, and occlusion scan data 430 may represent scan data obtained by scanning the patient's oral cavity with the upper jaw and the lower jaw occluded. In FIG. 4, the occlusion scan data 430 is illustrated as being obtained by scanning all of the patient's teeth; however, the embodiments are not limited thereto. The occlusion scan data used for occlusion alignment may not require all of the patient's teeth and may be obtained by scanning some of the patient's teeth. For example, the occlusion scan data used for occlusion alignment may be obtained by scanning 3 to 4 teeth in each of the upper jaw and the lower jaw. Particularly, the occlusion scan data may be obtained by scanning the teeth in the patient's molar region (posterior region). When the teeth in the posterior region are lost, the occlusion scan data may be obtained by scanning the teeth in the patient's canine region.

Because the upper-jaw scan data 410 is data obtained by scanning only the upper jaw in the patient's oral cavity and the lower-jaw scan data 420 is data obtained by scanning only the lower jaw in the patient's oral cavity, information about the occlusion surface when the upper jaw and the lower jaw are occluded, that is, the occlusion position relationship, may not be obtained from the upper-jaw scan data and the lower-jaw scan data. Thus, the occlusion position relationship may be obtained by aligning the upper-jaw scan data and the lower-jaw scan data with the occlusion scan data 430 obtained by scanning one or more teeth with the upper jaw and the lower jaw occluded.

Returning back to FIG. 3, in operation 320, the data processing apparatus 100 may identify a mobility tooth from at least one of the upper-jaw scan data or the lower-jaw scan data.

The mobility tooth may refer to a loose tooth among the teeth in the oral cavity, and because the loose tooth is not fixed and may move, the positions of the mobility tooth included in the scan data may be unreliable. For example, when a molar at the rearmost portion of the oral cavity in the patient's upper jaw is a mobility tooth, because it is not possible to ensure that the position of the molar, which is the mobility tooth, is accurate in the data obtained by scanning the upper jaw including the molar in a loose tooth state, it may be desirable not to use the mobility tooth in an occlusion alignment operation. Thus, according to the described embodiments, the data processing apparatus 100 may identify a mobility tooth from at least one of the upper-jaw scan data or the lower-jaw scan data.

According to an embodiment, the data processing apparatus 100 may identify a mobility tooth in the upper-jaw scan data, identify a mobility tooth in the lower-jaw scan data, or identify a mobility tooth in both the upper-jaw scan data and the lower-jaw scan data. Also, the data processing apparatus 100 may identify one or more mobility teeth.

According to an embodiment, the data processing apparatus 100 may identify a mobility tooth based on a user input. Particularly, the data processing apparatus 100 may provide a user interface for selecting a mobility tooth and may identify a mobility tooth through the user interface based on a user input for selecting one or more mobility teeth.

According to an embodiment, the data processing apparatus 100 may automatically recognize a mobility tooth by using various techniques.

According to an embodiment, the data processing apparatus 100 may automatically recognize a mobility tooth in a rule-based manner. For example, when the data processing apparatus 100 detects a tooth having a shape different from a general tooth shape, the data processing apparatus 100 may recognize the tooth as a mobility tooth. In this case, the data processing apparatus 100 may recognize, as a mobility tooth, a tooth that is greatly different in shape or position from the teeth included in a template model.

According to an embodiment, the data processing apparatus 100 may recognize a tooth with an excessive occlusion contact as a mobility tooth. For example, the data processing apparatus 100 may recognize, as a mobility tooth, a tooth whose contact area with the opposing tooth is greater than or equal to a threshold value.

According to an embodiment, the data processing apparatus 100 may recognize a mobility tooth by determining whether the gum is significantly lowered in the x-ray data obtained by photographing the oral cavity.

According to an embodiment, the data processing apparatus 100 may recognize a mobility tooth based on data through neural network learning based on artificial intelligence.

In operation 330, the data processing apparatus 100 may perform occlusion alignment of the upper-jaw scan data and the lower-jaw scan data by using the occlusion scan data excluding the identified mobility tooth. For example, as illustrated in FIG. 4, when a tooth 421 is identified as a mobility tooth in the lower-jaw scan data 420, the data processing apparatus 100 may prevent the teeth 421 corresponding to the mobility tooth from being used in an alignment operation by performing an operation of aligning other teeth than the tooth 421 among the teeth of the lower-jaw scan data 420 with the occlusion scan data 430.

The data processing apparatus 100 may perform occlusion alignment by using manual alignment or automatic alignment.

In the case of the automatic alignment, the data processing apparatus 100 may perform occlusion alignment by moving or rotating the upper-jaw scan data and the lower-jaw scan data until the upper-jaw scan data and the lower-jaw scan data are close to the position defined by the occlusion scan data. When the occlusion alignment is completed, the data processing apparatus 100 may store information representing the position relationship between the upper-jaw scan data and the lower-jaw scan data in the occlusal state.

As such, in most cases, the upper-jaw scan data and the lower-jaw scan data are automatically aligned with buccal occlusion scan data; however, when there is overocclusion or the inherent shape of the buccal occlusion surface is insufficient, the automatic alignment may fail. In this case, the occlusion alignment may be completed by using a manual alignment function.

In the manual alignment, the data processing apparatus 100 may perform occlusion alignment by displaying an alignment reference point in a jaw arch, receiving a user input for designating a corresponding alignment point in occlusion data, and matching the designated alignment point thereto.

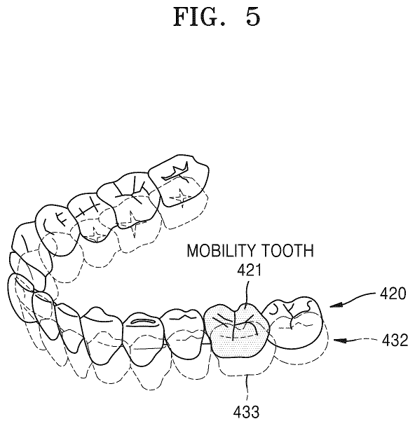

FIG. 5 is a reference diagram for describing an example of performing occlusion alignment excluding a mobility tooth, according to an embodiment.

Referring to FIG. 5, the data processing apparatus 100 may perform occlusion alignment by moving or rotating the lower-jaw scan data 420 until the lower-jaw scan data 420 is close to the position defined by the occlusion scan data, particularly, to scan data 432 corresponding to the lower jaw among the occlusion scan data 430. In this case, when the tooth 421 is identified as a mobility tooth in the lower-jaw scan data 420, the data processing apparatus 100 may find an occlusion alignment position by moving or rotating other tooth data than the mobility tooth 421 in the lower-jaw scan data 420 until the other tooth data is close to the position defined by the scan data 432. Because a tooth 433 included in the scan data 432 is also the same mobility tooth but is a moving tooth due to the property of the mobility tooth, because the shape or position of the tooth 421 may be different from that of the tooth 433, more reliable results may be obtained when alignment is performed excluding the mobility tooth 421.

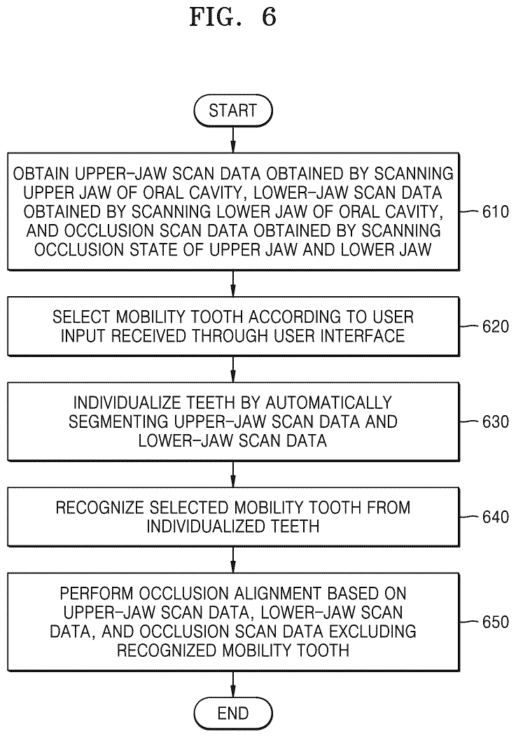

FIG. 6 is a flowchart illustrating an example of a process of a method of performing occlusion alignment of scan data, according to an embodiment.

Referring to FIG. 6, in operation 610, the data processing apparatus 100 may obtain upper-jaw scan data obtained by scanning the upper jaw of the oral cavity, lower-jaw scan data obtained by scanning the lower jaw of the oral cavity, and occlusion scan data obtained by scanning an occlusion state of the upper jaw and the lower jaw.

In operation 620, the data processing apparatus 100 may select a mobility tooth according to a user input received through a user interface. Particularly, the data processing apparatus 100 may provide a user interface for selecting a mobility tooth and may receive a user input for selecting one or more mobility teeth through the user interface.

In the example illustrated in FIG. 6, scan data is obtained in operation 610 and a mobility tooth is selected in operation 620; however, this is merely an example and does not represent a sequential relationship. Thus, scan data may be obtained after a mobility tooth is selected. Also, a mobility tooth selecting operation and a scan data obtaining operation may be simultaneously performed.

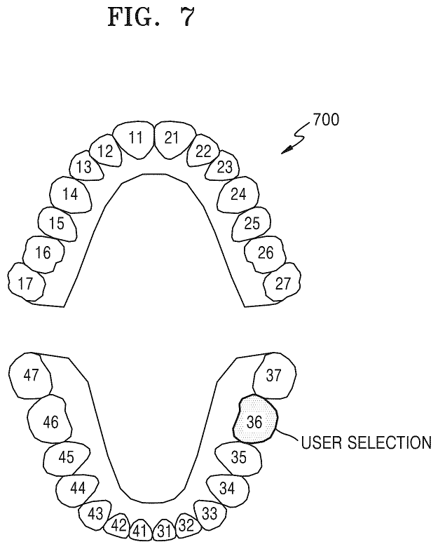

FIG. 7 illustrates an example of a user interface for selecting a mobility tooth, according to an embodiment.

Referring to FIG. 7, the data processing apparatus 100 may output a user interface 700 with a tooth number assigned to each of the upper-jaw teeth and the lower-jaw teeth. The user may select a mobility tooth by selecting one or more tooth numbers among the tooth numbers of the upper-jaw teeth and the lower-jaw teeth displayed on the user interface 700. The user may select one or more tooth numbers among the upper-jaw teeth, among the lower-jaw teeth, or among the upper-jaw teeth and the lower-jaw teeth.

Returning back to FIG. 6, in operation 630, the data processing apparatus 100 may individualize the teeth by automatically segmenting the upper-jaw scan data and the lower-jaw scan data. The upper-jaw scan data 410 and the lower-jaw scan data 420 obtained in operation 610 may be data obtained by imaging the entire surface of the patient's oral cavity and thus may be data in which one or more teeth and the gum surrounding the teeth are all lumped together. Thus, in order for the data processing apparatus 100 to obtain individual information about the teeth included in the upper-jaw scan data 410 and the lower-jaw scan data 420, an operation of individualizing the teeth included in the tooth area may be required.

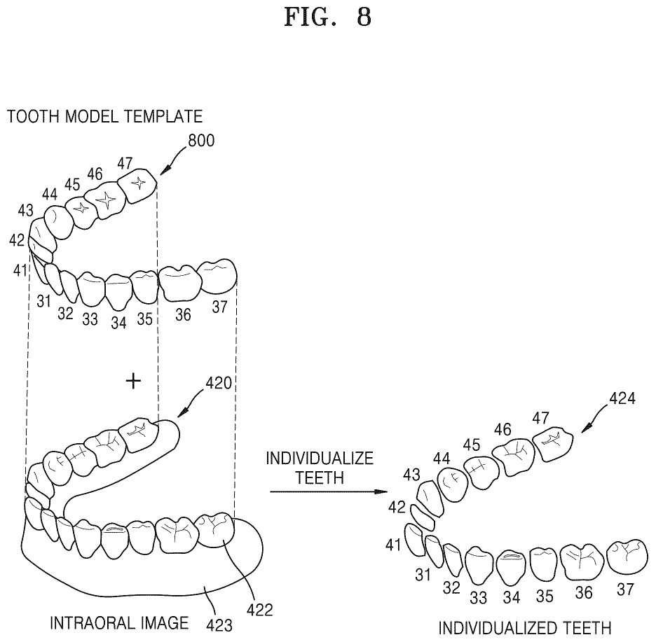

FIG. 8 is a reference diagram for describing a method of individualizing teeth, according to an embodiment.

The lower-jaw scan data 420 may have a polygonal mesh structure because adjacent apexes in a point cloud whose apexes are coordinates of points obtained by scanning an object by an intraoral scanner are polygonized. The polygon constituting the mesh structure may be a triangle, a square, a pentagon, or the like and may be a triangle in an embodiment. As such, in the lower-jaw scan data 420 having a polygonal mesh structure generated by the data processing apparatus 100, teeth 422 and a gum 423 may not be separated from each other and the teeth 422 may also be recognized as a tooth mass in which a plurality of teeth are not separated from each other. Alternatively, according to another embodiment, because the data processing apparatus 100 separates the teeth and a gum area from each other in a 2D image obtained in a scan process by using artificial intelligence, the lower-jaw scan data 420 generated by the data processing apparatus 100 may be automatically separated into a tooth mass and the gum.

A tooth model template 800 may represent template model data in which the teeth have ideal shapes and are arranged at ideal positions and each tooth is numbered. For example, the template teeth of the tooth model template 800 may be respectively numbered from 31 to 37 and from 41 to 47.

The data processing apparatus 100 may separate the lower-jaw scan data 420 into the teeth 422 and the gum 423 by using artificial intelligence technology, curvature distribution, or the like. Also, the data processing apparatus 100 may obtain individualized teeth 424 by individualizing the teeth of the lower-jaw scan data by performing a data processing operation of aligning the teeth 422 of the lower-jaw scan data 420 by using the tooth model template 800. Individualizing the teeth may mean separating the teeth from the gum in the scan data and also obtaining information about each of the teeth. The information about each tooth may include information about the shape of each tooth, information about the position of each tooth, and information about the number of each tooth. Individualization of the teeth may also be referred to as segmentation of the teeth, fragmentation of the teeth, or the like. By individualizing the teeth of the scan data as such, the data processing apparatus 100 may delete or move each tooth or insert an additional tooth by using the individualized teeth 424. According to an embodiment, when aligning a tooth model template 200 with the teeth 422, the data processing apparatus 100 may use various automatic alignment algorithms, for example, an iterative closest point (ICP) algorithm.

Returning back to FIG. 6, in operation 640, the data processing apparatus 100 may recognize the mobility tooth selected in operation 620, from the teeth individualized in operation 630. For example, when the user selects a mobility tooth corresponding to the tooth number 36 in operation 620, the data processing apparatus 100 may recognize a tooth corresponding to the tooth number 36 from the teeth 424 individualized in operation 630.

In operation 650, the data processing apparatus 100 may perform occlusion alignment of the upper-jaw scan data and the lower-jaw scan data by using the occlusion scan data excluding the recognized mobility tooth. The occlusion alignment may be the same as described with reference to FIG. 3.

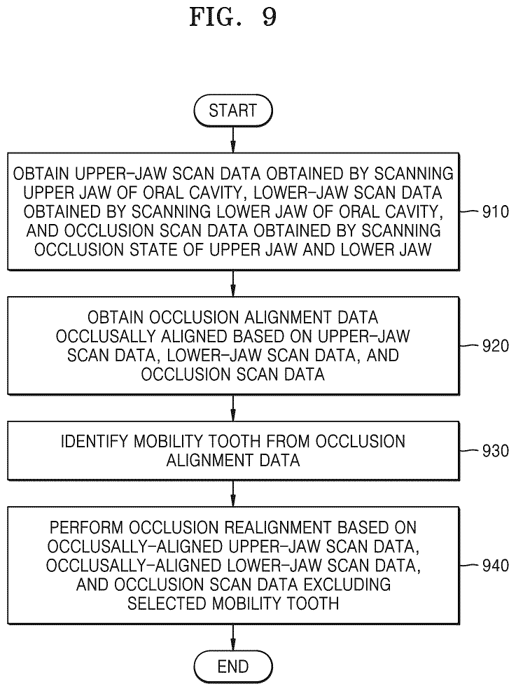

FIG. 9 is a flowchart illustrating an example of a process of a method of performing occlusion alignment of scan data, according to an embodiment.

Referring to FIG. 9, in operation 910, the data processing apparatus 100 may obtain upper-jaw scan data obtained by scanning the upper jaw of the oral cavity, lower-jaw scan data obtained by scanning the lower jaw of the oral cavity, and occlusion scan data obtained by scanning an occlusion state of the upper jaw and the lower jaw.

In operation 920, the data processing apparatus 100 may perform occlusion alignment based on the upper-jaw scan data, the lower-jaw scan data, and the occlusion scan data and obtain occlusion alignment data. That is, the data processing apparatus 100 may obtain occlusally-aligned upper-jaw scan data and occlusally-aligned lower-jaw scan data by occlusally aligning the upper-jaw scan data and the lower-jaw scan data by using the occlusion scan data based on the upper-jaw scan data and the lower-jaw scan data, and the occlusion scan data including the mobility tooth.

In operation 930, the data processing apparatus 100 may select a mobility tooth from the occlusion alignment data. Particularly, the data processing apparatus 100 may identify a mobility tooth from the occlusally-aligned upper-jaw scan data and the occlusally-aligned lower-jaw scan data obtained in operation 920.

The data processing apparatus may identify a mobility tooth by using various techniques.

According to an embodiment, the data processing apparatus 100 may identify a mobility tooth based on a user input. Particularly, the data processing apparatus 100 may provide a user interface including the occlusally-aligned upper-jaw scan data and the occlusally-aligned lower-jaw scan data for selecting a mobility tooth and may identify a mobility tooth through the user interface based on a user input for selecting one or more mobility teeth.

According to an embodiment, the data processing apparatus 100 may automatically recognize a mobility tooth.

According to an embodiment, the data processing apparatus 100 may automatically recognize a mobility tooth in a rule-based manner. For example, when the data processing apparatus 100 detects a tooth having a shape different from a general tooth shape, the data processing apparatus 100 may recognize the tooth as a mobility tooth. In this case, the data processing apparatus 100 may recognize, as a mobility tooth, a tooth that is greatly different in shape or position from the teeth included in a template model.

According to an embodiment, the data processing apparatus 100 may recognize a tooth with an excessive occlusion contact as a mobility tooth. For example, the data processing apparatus 100 may recognize, as a mobility tooth, a tooth whose contact area is greater than or equal to a threshold value.

According to an embodiment, the data processing apparatus 100 may recognize a mobility tooth by determining whether the gum is significantly lowered in the x-ray data obtained by photographing the oral cavity.

According to an embodiment, the data processing apparatus 100 may recognize a mobility tooth based on data through neural network learning based on artificial intelligence.

In operation 940, the data processing apparatus 100 may perform occlusion realignment of the occlusally-aligned upper-jaw scan data and the occlusally-aligned lower-jaw scan data based on the occlusion scan data excluding the selected mobility tooth. That is, the data processing apparatus 100 may obtain more precise occlusion alignment results by performing, in operation 920, primary occlusion alignment by using the upper-jaw scan data, the lower-jaw scan data, and the occlusion scan data without identifying the mobility tooth and then performing, in operation 940, occlusion realignment on the primarily occlusion-aligned scan data excluding the mobility tooth.

When the mobility tooth is selected before the occlusion scan from the upper-jaw scan data or the lower-jaw scan data by the user by using a tool such as a brush, occlusion alignment may be performed excluding the mobility tooth immediately after obtaining the occlusion scan data, and thus, in this case, occlusion realignment may not be required as in the example illustrated in FIG. 6. The example illustrated in FIG. 9 may be applied when the mobility tooth is selected after primary alignment is performed by using the upper-jaw scan data, the lower-jaw scan data, and the occlusion scan data without selecting the mobility tooth.

Examples of selecting the mobility tooth to be excluded from the occlusion alignment have been described in the above embodiments; however, according to another embodiment, an input for actively selecting teeth to be used for occlusion alignment may be received from the user.

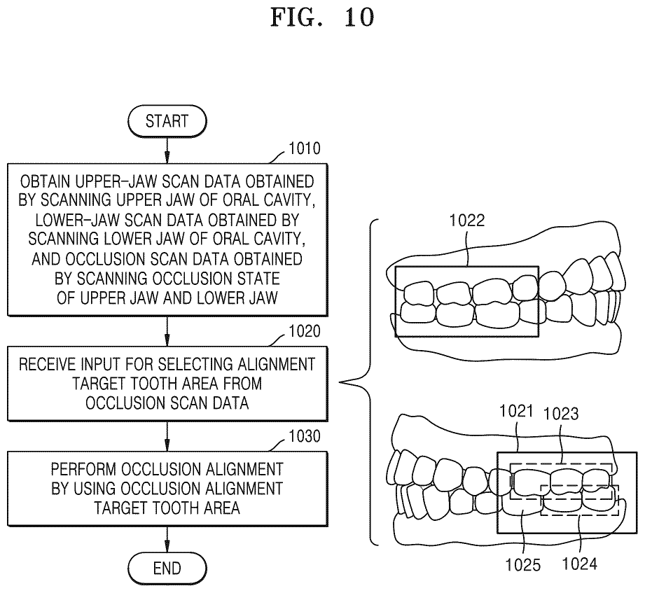

FIG. 10 is a flowchart illustrating an example of a process of a method of performing occlusion alignment of scan data, according to an embodiment.

Referring to FIG. 10, in operation 1010, the data processing apparatus 100 may obtain upper-jaw scan data obtained by scanning the upper jaw of the oral cavity, lower-jaw scan data obtained by scanning the lower jaw of the oral cavity, and occlusion scan data obtained by scanning an occlusion state of the upper jaw and the lower jaw.

In operation 1020, the data processing apparatus 100 may receive an input for selecting an occlusion alignment target tooth area in the occlusion scan data. Particularly, the data processing apparatus 100 may select an occlusion alignment target tooth area 1021 at a first position of the occlusion scan data, for example, on the right side and may select an occlusion alignment target tooth area 1022 at a second position of the occlusion scan data, for example, on the left side. Each of the occlusion alignment target tooth areas 1021 and 1022 may include one or more upper-jaw teeth and one or more lower-jaw teeth. In this case, the data processing apparatus 100 may select the occlusion alignment target tooth areas 1021 and 1022 such that each of the occlusion alignment target tooth areas 1021 and 1022 does not include a mobility tooth. For example, the occlusion alignment target tooth area 1021 may include an upper-jaw tooth area 1023 including three upper-jaw teeth and a lower-jaw tooth area 1024 including two lower-jaw teeth but may not include a lower-jaw mobility tooth 1025.

According to an embodiment, the data processing apparatus 100 may select an occlusion alignment target tooth area excluding the mobility tooth according to a user input using a brush or the like. Alternatively, the data processing apparatus 100 may select an occlusion alignment target tooth area excluding the mobility tooth according to the own determination of the data processing apparatus 100.

In operation 1030, the data processing apparatus 100 may perform an occlusion alignment operation by using the occlusion alignment target tooth area. The data processing apparatus 100 may obtain occlusally-aligned upper-jaw scan data and occlusally-aligned lower-jaw scan data by occlusally aligning the upper-jaw scan data and the lower-jaw scan data by using the occlusion alignment target tooth area selected in the occlusion scan data.

An intraoral image processing method according to an embodiment of the present disclosure may be embodied in the form of program commands executable through various computer means and then may be recorded on a computer-readable recording medium. Also, an embodiment of the present disclosure may be a computer-readable storage medium having recorded thereon one or more programs including at least one instruction for executing the intraoral image processing method.

The computer-readable storage medium may include program instructions, data files, and data structures either alone or in combination. Here, examples of the computer-readable storage medium may include magnetic media such as hard disks, floppy disks, and magnetic tapes, optical media such as CD-ROMs and DVDs, magneto-optical media such as floptical disks, and hardware devices such as ROMs, RAMs, and flash memories that are configured to store and execute program instructions.

Here, a machine-readable storage medium may be provided in the form of a non-transitory storage medium. Here, the ‘non-transitory storage medium’ may mean that the storage medium is a tangible device. Also, the ‘non-transitory storage medium’ may include a buffer in which data is temporarily stored.

According to an embodiment, the intraoral image processing method according to various embodiments described herein may be included and provided in a computer program product. The computer program product may be distributed in the form of a machine-readable storage medium (e.g., a compact disc read only memory (CD-ROM)). Alternatively, the computer program product may be distributed (e.g., downloaded or uploaded) online through an application store (e.g., Play Store) or directly between two user devices (e.g., smart phones). Particularly, the computer program product according to the described embodiments may include a storage medium having recorded thereon a program including at least one instruction for performing the intraoral image processing method according to the described embodiments.

Although embodiments have been described above in detail, the scope of the present disclosure is not limited thereto and various modifications and improvements made by those of ordinary skill in the art by using the basic concept of the present disclosure defined in the following claims are also included in the scope of the present disclosure.

1. An intraoral image processing method of a data processing apparatus, the intraoral image processing method comprising:
obtaining upper-jaw scan data obtained by scanning an upper jaw of an oral cavity, lower-jaw scan data obtained by scanning a lower jaw of the oral cavity, and occlusion scan data obtained by scanning an occlusion state of the upper jaw and the lower jaw;
selecting a mobility tooth; and
performing occlusion alignment of the upper-jaw scan data and the lower-jaw scan data based on the occlusion scan data excluding the selected mobility tooth.

2. The intraoral image processing method of claim 1, wherein the selecting of the mobility tooth comprises selecting the mobility tooth according to a user input received through a user interface.

3. The intraoral image processing method of claim 2, wherein the performing of the occlusion alignment comprises:
individualizing teeth of the upper-jaw scan data and teeth of the lower-jaw scan data;
recognizing the selected mobility tooth from the individualized teeth; and
performing occlusion alignment of the upper-jaw scan data and the lower-jaw scan data by using the occlusion scan data excluding the recognized mobility tooth.

4. The intraoral image processing method of claim 1, wherein the selecting of the mobility tooth comprises:
obtaining occlusion alignment upper-jaw scan data and occlusion alignment lower-jaw scan data by performing occlusion alignment of the upper-jaw scan data and the lower-jaw scan data by using the occlusion scan data; and
selecting the mobility tooth from the occlusion alignment upper-jaw scan data and the occlusion alignment lower-jaw scan data, and
the performing of the occlusion alignment comprises performing occlusion realignment of the occlusion alignment upper-jaw scan data and the occlusion alignment lower-jaw scan data by using the occlusion scan data excluding the selected mobility tooth.

5. The intraoral image processing method of claim 4, wherein the selecting of the mobility tooth from the occlusion alignment upper-jaw scan data or the occlusion alignment lower-jaw scan data comprises selecting the mobility tooth based on a user input for selecting the mobility tooth through a user interface with the occlusion alignment upper-jaw scan data or the occlusion alignment lower-jaw scan data displayed therein, or automatically selecting the mobility tooth by the data processing apparatus.

6. A data processing apparatus comprising:
a processor; and a memory,
wherein the processor is configured to execute one or more instructions stored in the memory to:
obtain upper-jaw scan data obtained by scanning an upper jaw of an oral cavity, lower-jaw scan data obtained by scanning a lower jaw of the oral cavity, and occlusion scan data obtained by scanning an occlusion state of the upper jaw and the lower jaw;
select a mobility tooth; and
perform occlusion alignment of the upper-jaw scan data and the lower-jaw scan data by using the occlusion scan data excluding the selected mobility tooth.

7. The data processing apparatus of claim 6, wherein the processor is further configured to execute the one or more instructions stored in the memory to select the mobility tooth according to a user input received through a user interface.

8. The data processing apparatus of claim 7, wherein the processor is further configured to execute the one or more instructions stored in the memory to:
individualize teeth of the upper-jaw scan data and teeth of the lower-jaw scan data;
recognize the selected mobility tooth from the individualized teeth; and
perform occlusion alignment of the upper-jaw scan data and the lower-jaw scan data by using the occlusion scan data excluding the recognized mobility tooth.

9. The data processing apparatus of claim 6, wherein the processor is further configured to execute the one or more instructions stored in the memory to:
obtain occlusion alignment upper-jaw scan data and occlusion alignment lower-jaw scan data by performing occlusion alignment of the upper-jaw scan data and the lower-jaw scan data by using the occlusion scan data;
select the mobility tooth from the occlusion alignment upper-jaw scan data and the occlusion alignment lower-jaw scan data; and
perform occlusion realignment of the occlusion alignment upper-jaw scan data and the occlusion alignment lower-jaw scan data by using the occlusion scan data excluding the selected mobility tooth.

10. The data processing apparatus of claim 9, wherein the processor is further configured to execute the one or more instructions stored in the memory to:
select the mobility tooth from the occlusion alignment upper-jaw scan data or the occlusion alignment lower-jaw scan data; and
select the mobility tooth based on a user input for selecting the mobility tooth through a user interface with the occlusion alignment upper-jaw scan data or the occlusion alignment lower-jaw scan data displayed therein, or automatically select the mobility tooth by the data processing apparatus.

11. A computer-readable recording medium having recorded thereon a program implemented to execute an intraoral image processing method by a processor of a data processing apparatus, wherein the intraoral image processing method comprises:
obtaining upper-jaw scan data obtained by scanning an upper jaw of an oral cavity, lower-jaw scan data obtained by scanning a lower jaw of the oral cavity, and occlusion scan data obtained by scanning an occlusion state of the upper jaw and the lower jaw;
selecting a mobility tooth; and
performing occlusion alignment of the upper-jaw scan data and the lower-jaw scan data by using the occlusion scan data excluding the selected mobility tooth.
